(19) United States
(12) Patent Application Publication
(10) Pub. No.: US 2025/0275911 A1
(43) Pub. Date: Sep. 4, 2025

(54) Cosmetic Composition Containing an Extract of Gentiana and Leontopodium and/or Centella

(71) Applicant: WELEDA AG, Arlesheim (CH)

(72) Inventors: Katarzyna Hänni-Ciunel, Magden (CH); Alicia Idoux, Bättwil (CH); Petra Bach, Stuttgart (DE); Dagmar Bässler, Schwäbisch Gmünd (DE); Bernhard Irrgang, Menziken (CH); Eva-Maria Lohmüller, Schwäbisch Gmünd (DE); Meike Schmitt, Emmendingen (DE)

(21) Appl. No.: 18/705,158

(22) PCT Filed: Oct. 27, 2022
(86) PCT No.: PCT/EP2022/080153

§ 371 Date: Apr. 26, 2024

(30) Foreign Application Priority Data
Oct. 27, 2021 (EP) 21204968.8

(51) Int. Cl. A61K 8/9789; A61Q 17/04; A61Q 19/08

(52) U.S. Cl. CPC A61K 8/9789; A61Q 17/04; A61Q 19/08; A61K 2800/5922

(57) ABSTRACT

The invention relates to a cosmetic composition comprising Gentiana (gentian) and Leontopodium (edelweiss) and/or Centella (Indian pennywort), and its use, in particular as an anti-ageing/anti-wrinkle agent.

Gentiana, Leontopodium and Centella are able to delay skin ageing by surprisingly inhibiting the protease MMP-1, which is responsible for the breakdown of collagen in the dermis, and stimulating collagen-1 expression in connective tissue.

The invention relates to a cosmetic composition comprising Gentiana, Leontopodium and/or Centella, and its use, in particular as an anti-ageing agent.

Weleda AG conducts research into beneficial natural plant substances for cosmetics using modern and anthroposophical methods.

The plant genus Gentiana (gentian) from the gentian family (Gentianaceae) with approx. 400 species, the above-ground parts of the plant or the roots contain characteristic and significant quantities of bitter substances from the plant substance group iridoids, in particular secoiridoid glycosides such as swertiamarin, amarogentin and gentiopicroside, as well as flavonoids, in particular iso-orientin, iso-vitexin, iso-orientin-4′-O-glucoside and iso-saponarin. They are mainly found in the high mountains. The best known are yellow gentian (Gentiana lutea) and various species of “blue gentian” with characteristic blue calyx flowers of the species Gentiana alpina, Gentiana acaulis, Gentiana clusii, Gentiana brachyphylla, Gentianan orbicularis and Gentiana septemfida var.

The genus Leontopodium (edelweiss) is also found in the high mountains and the aerial parts of the plant are characterized by leontopodic acids (edelweiss acids) and chlorogenic acid, lignans, especially leoligin and 5-methoxyleoligin, flavonoids such as luteolin-4′-O-glucoside, apigenin-7-O-glucoside and luteolin and a high content of tannins. The most well-known species are Leontopodium alpinum (so-called alpine edelweiss), Leontopodium nivale and Leontopodium palibinianum.

Centella (Indian pennywort, also known as Asiatic pennywort, tiger grass or gotu kola) is a plant genus native to the tropics and subtropics. Characteristic ingredients in the aerial parts of the plant are triterpene saponins, in particular asiaticosides and madecasosides, asiatic acid, madecassic acid and flavonol glycosides. The best known species is Centella asiatica.

As the largest organ in the human body, the skin has numerous vital functions and forms the interface with the environment. It protects the human organism from physical and mechanical stress (heat, cold, UV rays, impacts, shear forces) and from allergens, chemicals and microorganisms penetrating the body (barrier from the outside in). On the other hand, it reduces water loss through the skin (barrier from the inside out). The main functions are therefore the barrier function against damaging external influences and the regulation of the cutaneous water balance.

The barrier is similar in structure to a brick wall. Its horny cells form the “bricks” of the masonry, while the “cement” in between is a complex hydrolipid film consisting of lipids such as ceramides, but also proteins and enzymes. The interaction of these individual components can easily be disrupted, which affects the barrier function. One factor here is age—with age, the activities of sebaceous and sweat glands and the ability of cell regeneration, among other things, decline.

Skin ageing per se is a complex process that is influenced by various factors that interact with each other and affect the skin layers in different ways.

Intrinsic skin ageing refers to the processes that take place in all cells of the body and is referred to as biological ageing. Extrinsic factors are external influences on the skin. The human environment plays a causal role here. In addition to unhealthy stress, smoking, poor diet and alcohol consumption, UV radiation has the greatest negative impact on the skin.

Skin ageing takes place in the epidermis in the form of slower cell metabolism and lower lipid production. The consequences: the barrier function deteriorates, causing the skin to dry out and become rougher, resulting in fine lines and wrinkles, especially on the face.

The dermis consists mainly of connective tissue cells, known as fibroblasts, and connective tissue fibers made of collagen and elastin. In young skin, both types of fibers work together perfectly: Collagen makes the tissue stable and tensile, while the rubbery elastin ensures that it remains stretchy. In the dermis, the skin constantly builds up and breaks down collagen. With increasing age, the degradation processes predominate, i.e. the skin loses collagen and elastin. The connective tissue cells in the dermis also produce less and less collagen over time. As a result, fine lines and wrinkles appear more quickly, especially on the face, and the skin becomes thinner.

The inner skin layer (subcutis) consists mainly of fat cells and special collagen fibers that serve as connecting elements. This layer of skin is responsible for storing energy, cushioning and insulating the body. Processes also take place here as a result of skin ageing, the number and size of lipid cells decreases, which also leads to thinner skin and loss of volume.

The process of skin ageing per se cannot be stopped, but the process of extrinsic ageing can be slowed down. In addition to a healthy lifestyle and appropriate skin care with suitable skin care products, skin ageing can be at least partially delayed.

As a direct active ingredient in cosmetics, collagen has good moisturizing properties. Collagen type I in particular is a fibrillar collagen, the most common type of collagen, and is important for the firmness of the skin's connective tissue.

However, collagen molecules are far too large for application to actually penetrate the skin with an intact barrier. Active ingredients that can stimulate collagen synthesis in the skin and/or inhibit the breakdown of the body's own collagen are therefore desirable for cosmetics.

In the prior art, extracts of Gentiana, Leontopodium or Centella are described for use in cosmetics.

CN 111631961 A discloses a moisturizing mask for the face, which may include a gentian root extract (gentian root 0.2-0.5 w/w) together with alpine edelweiss (0.5-2 w/w) and Centella asiatica (0.5-1.5 w/w).

Surprisingly, the inventors found that extracts of i.) Gentiana and Leontopodium, or ii.) Gentiana and Centella or iii.) Gentiana and Leontopodium and Centella have an influence on the amount of collagen type 1 in the extracellular matrix and show strong synergistic effects.

In two different in-vitro test models (assays), corresponding synergistic significant effects of the extracts of Gentiana, Leontopodium and Centella were demonstrated:

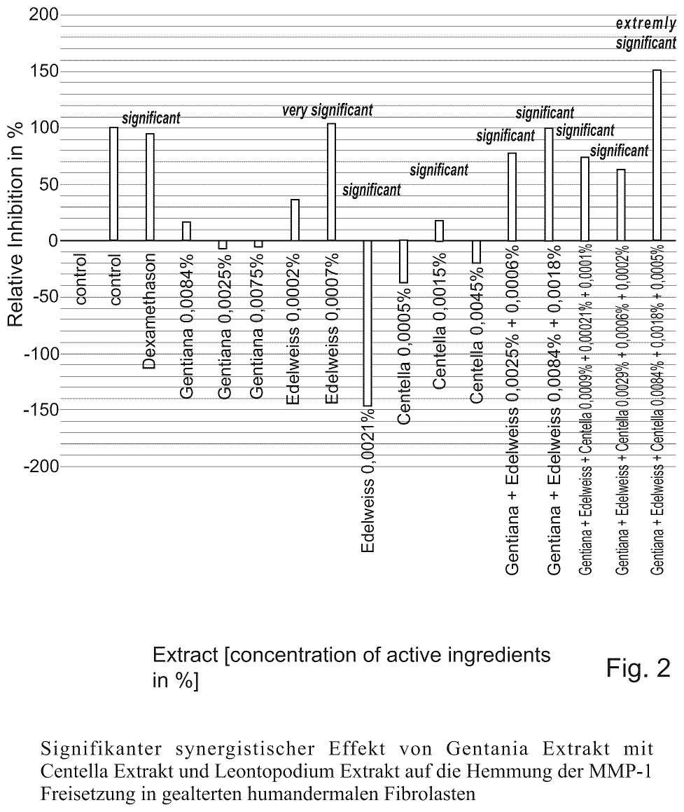

On the one hand, there is a significant synergistic effect both 1) on collagen type I expression in the extracellular matrix in human dermal fibroblasts and 2) on the inhibition of the release of matrix melloproteinase 1 (MMP-1), an important degradation enzyme of collagen 1. This is all the more surprising as Gentiana, Centella and especially Edelweiss as single substances or single extracts even show opposite effects (see FIG. 2), so that even a positive-additive effect in a combination was not to be expected.

Extrinsic factors, above all sunlight, induce enzymes in the skin that break down components of the intercellular matrix, such as collagen and elastin, including MMP-1. Cigarette smoke and other environmental contaminants have also been shown in various epidemiological studies and ultrastructurally to induce various matrix metalloproteinases, such as MMP-1, which thus significantly reduces collagen biosynthesis (Thieme, Haut und Alter, Effendy, Isaak et. al. 2005, Physiology of skin ageing; Araviiskaia et al. The impact of airborne pollution on skin, JEADV (2019) 10.1111/jdv.15583, https://doi.org/10.1111/jdv.15583).

Consequently, the combinations of the extracts of i.) Gentiana and Leontopodium, or ii.) Gentiana and Centella or iii.) Gentiana and Leontopodium and Centella are particularly suitable for delaying the skin ageing process by increasing the expression of type I collagen and inhibiting MMP-1.

Particularly in a suitable galenic formulation, they can therefore help to delay skin ageing (anti-ageing) and therefore also delay the formation of wrinkles (anti-wrinkle). A stress effect caused by pollution or short-wave light is also reduced, or at least delayed (anti-pollution, light protection, especially blue light and IR, UV protection).

The present invention is therefore based on the task of providing a cosmetic composition for topical use which can in particular delay the skin ageing process.

The object is accomplished by a cosmetic composition comprising extracts of i.) Gentiana and Leontopodium or ii.) Gentiana and Leontopodium and Centella or iii.) Gentiana and Leontopodium and Centella, or by the imparted technical teaching of at least one patent claim.

Therefore, the invention relates to such a cosmetic composition and an agent thereof, in particular an anti-aging agent, an anti-wrinkle agent, an anti-pollution agent, a light protection agent, in particular a blue light and IR, UV protection agent.

In a preferred embodiment, the extracts of Gentiana, Leontopodium and Centella are obtained from the plant material or drug in the hot or cold by means of an extracting agent which contains water and/or at least one organic solvent, in particular C1-C4 alcohols, preferably ethanol. Water can also be used as an extracting agent. The extracts can be obtained, for example, by extraction, decoction, percolation or maceration from the preferably dried plant parts. A common extraction is, for example, an aqueous-ethanolic extraction, in particular a mixture of water/ethanol (50:50, in particular in the range of 80:20 or 20:80), e.g. at 15 to 80 degrees Celsius and normal pressure.

The extracts can be stabilized with polyhydric alcohols such as glycerol or glycols and/or with the help of antimicrobial additives.

A preferred preparation of a Gentiana extract is the decoction of the aerial parts of the plant, preferably with an ethanol-water mixture. After removal of the solvent, the fluid extract is absorbed in glycerol and/or glycols. Furthermore, blue gentian is preferred, especially the species Gentiana septemfida and Gentiana acaulis.

A preferred preparation of a Leontopodium extract is the decoction of the aerial parts of the plant, in particular the flowers and leaves, preferably with an ethanol-water mixture. After removal of the solvent, the fluid extract can be absorbed in glycerol and/or glycols. Furthermore, the species Leontopodium alpinum is preferred.

A preferred preparation of a Centella extract is the maceration of the aerial parts of the plant, preferably with an ethanol-water mixture. The extract is filtered and can be used directly. The species Centella asiatica is also preferred.

In a further preferred embodiment, the weight ratio in w/w (m/m) of the extracts of Gentiana, Leontopodium and Centella in a cosmetic composition is as follows: Gentiana:Leontopodium to 2.5-4:1 (w/w) or Gentiana:Centella to 2.5-4:1 (w/w) and Gentiana:Leontopodium:Centella to 2.5-4:0.5-1:0.5-1 (w/w/w).

Furthermore, it is preferred that the amounts by weight in a cosmetic composition contain more extracts in % by weight of Gentiana than Leontopodium and/or Centella.

Furthermore, it is preferred that the amounts by weight in a cosmetic composition of Gentiana, Leontopodium and Centella are each more than 0.5% by weight, preferably 1-2% by weight, 1-5% by weight is particularly preferred.

The stated weights of the above-mentioned extracts refer to the dry weight with residual moisture.

In addition to these active ingredients, an advantageous formulation comprises further auxiliaries and additives as well as care substances such as oils, in particular vegetable seed oils. An exemplary composition is given in Table 1 (Example 1), indicating the function of the ingredients and the amount used in % by weight relative to the total amount.

In a further embodiment, the cosmetic or dermatological composition according to the invention may be in the form of a gel, cream or lotion for topical use.

Emulsifiers can be advantageously selected from the group of non-ionic, anionic, cationic or amphoteric emulsifiers.

Emulsifiers produced from natural substances are preferred according to the invention, for example sugar esters based on fatty acids esterified with sucrose from natural oils, which are present in varying proportions as mono-, di- or even tri-esters and/or based on the amino acid glutamine, which is esterified with coconut oil fatty acids, for example. In addition, other W/O or O/W emulsifiers, e.g. based on ethylene oxide, can also be used.

The cosmetic compositions according to the invention may further contain cosmetic excipients as are commonly used in such preparations, e.g. antioxidants, vitamins, natural and/or synthetic fragrances, preservatives, dyes, pigments which have a colouring effect, thickening agents, rheological additives of mineral origin, surface-active substances, emollients, or other customary components of a cosmetic formulation such as alcohols, polyols, polymers, foam stabilizers, electrolytes, organic solvents.

In a further particular embodiment, the composition according to the invention preferably comprises naturally occurring and/or near-natural or nature-identical ingredients.

The following examples of embodiments and figures serve to illustrate the invention without limiting the invention to these examples.

### EXAMPLE 1

TABLE 1
Cosmetic preparation for topical application

| | | | Quantity |
|---|---|---|---|
| | | | in % by |
| no. | INCI | Function | weight |
| 1 | Hectorite | Thickener | 0.5-1.3% |
| 2 | Glycerine | Humectant | 7.8-12.2% |
| 3 | Aqua | Solvent | ad 100 |
| 4 | Betaine | Humectant | 0.5-1.3% |
| 5 | Citric Acid | Stabilizer | 0.1-0.4% |
| 6 | Sucrose Polystearate; | Emulsifier | 2.1-3.9% |
| | Cetyl Palmitate | | |
| 7 | Cetearyl Alcohol | Emulsifier | 2.1-3.9% |
| 8 | Olea Europaea Fruit Oil | Skin-caring oil | 6.3-9.5% |
| | | component | |
| 9 | Helianthus Annuus Seed Oil | Skin-caring oil | 6.3-9.5% |
| | | component | |
| 10 | Ricinus Communis Seed Oil | Skin-caring oil | 2.1-4.8% |
| | | component | |
| 11 | Sodium Stearoyl Glutamate | Emulsifier | 0.1-0.4% |
| 12 | Xanthan Gum | Thickener | 0.5-1.3% |
| 13 | Silica | Thickener | 0.5-1.3% |
| 14 | Pentylene Glycol | Solvent | 1.5-4.8% |
| 15 | Glyceryl Caprylate | Emulsifier | 0.1-0.4% |
| 16 | Tocopherol | Antioxidant | 0.04-0.07% |
| 17 | Perfume | Fragrance | 0.3-1.5% |
| 18 | Gentiana septemfida/ | Active ingredient | 0.5-5% |
| | acaulis Extract | | |
| 19 | Centella Asiatica Extract | Active ingredient | 0.5-2% |
| 20 | Leontopodium Alpinum | Active ingredient | 0.5-2% |
| | Extract | | |

### EXAMPLE 1A

### Collagen Type I Expression in Human Dermal Fibroblasts (Fixed Cells)

### Assay Determination

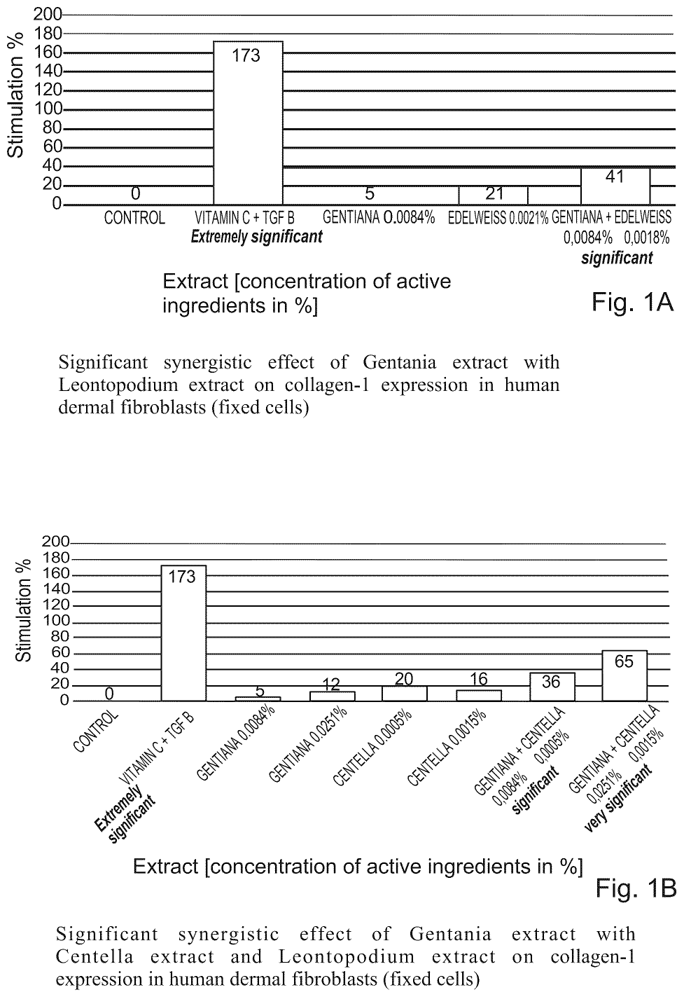

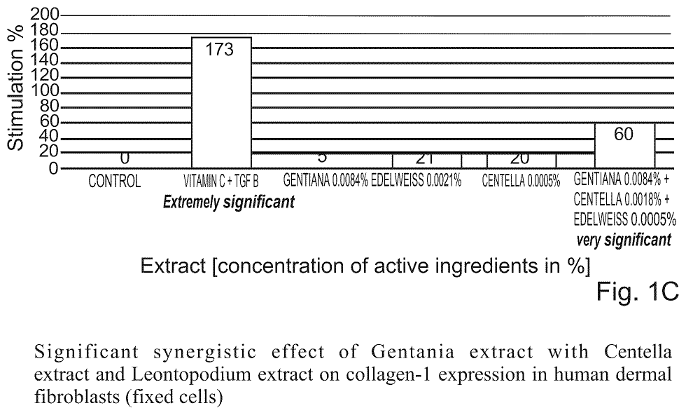

See FIG. 1A.

### Measurement of Two Extracts—Gentian and Edelweiss—Individually and in Combination.

Positive control: vitamin C+TGF β, negative control: assay medium without active substance, culture/assay medium: DMEM supplemented.
Incubation: 72 h, 96-microtiter plates.
In situ immunofluorescence labeling: anti-collagen 1/GAR Alexa 488/Hoechst 33258 (bis-benzimide).
Image analysis: INCell Analyzer™ 2200.
Number of determinations per parameter n=3

### Use Concentration of the Extracts:

Single: Gentian 15% extract, diluted to 0.0084%
Single: Edelweiss 7% extract, diluted to 0.0021%
Combination: Gentian 15% extract, diluted to 0.0084%+Edelweiss 7% extract, diluted to 0.00182%

### EXAMPLE 1B

### Collagen-1 Expression in Human Dermal Fibroblasts (Fixed Cells)

### Assay Determination

See FIG. 1B.

### Measurement of 2 Extracts—Gentian and Centella—Individually and in Combination

Positive control: vitamin C+TGF β, negative control: assay medium without active substance
Culture/assay medium: DMEM supplemented
Incubation: 72 h, 96-microtiter plates
In situ immunofluorescence labeling: anti-collagen 1/GAR Alexa 488/Hoechst 33258 (bis-benzimide)
Image analysis: INCell Analyzer™ 2200
Number of determinations per parameter n=3

### Use Concentration of the Extracts:

Single: Gentian 15% extract, diluted to 0.0084%/0.0251%
Single: Centella 9% extract, diluted to 0.0005%/0.0015%
Combination: gentian 15% extract, diluted to 0.0084%/0.0251%+centella 9% extract, diluted to 0.0005%/0.0015%

### EXAMPLE 1C

### Collagen-1 Expression in Human Dermal Fibroblasts (Fixed Cells)

### Assay Determination

See FIG. 1C.

### Measurement of 3 Extracts—Gentian, Edelweiss and Centella—Individually and in Combination

Positive control: vitamin C+TGF β, negative control: assay medium without active substance
Culture/assay medium: DMEM supplemented
Incubation: 72 h, 96-microtiter plates
In situ immunofluorescence labeling: anti-collagen 1/GAR Alexa 488/Hoechst 33258 (bis-benzimide)
Image analysis: INCell Analyzer™ 2200
Number of determinations per parameter n=3

### Use Concentration of the Extracts:

Single: Gentian 15% extract, diluted to 0.0084%
Single: Edelweiss 7% extract, diluted to 0.0021%
Single: Centella 9% extract, diluted to 0.0005%/0.0015%
Gentian 15% extract, diluted to 0.0084%+Edelweiss 7% extract, diluted to 0.0018%+Centella 9% extract, diluted to 0.0005%

The dual combination of Edelweiss+Centella extracts showed no synergistic effects on stimulation.

### EXAMPLE 2

### Inhibition of Matrix Metalloproteinase (MMP-1) in Aged Human Dermal Fibroblasts

### Assay Determination

See FIG. 2.

### Measurement of 3 Extracts—Gentian, Edelweiss and Centella—Individually and in Combinations of 2 and 3

Cell type: P8-NHDF (as control) and P18-F (“aged”) fibroblasts
Culture/assay medium: DMEM supplemented
Culture conditions: 37° C., 5° CO2
Incubation: 72h, 96-microtiter plates
Positive control: Dexamethasone 1 μM
Negative control: assay medium without verum
MMP-1 release detection: ELISA kit, Human total MMP-1 DuoSet, R&D Systems, ref. DY901B

### Use Concentration of the Extracts:

Single: Gentian 15%, diluted to 0.0084%/0.025%/0.075% e
Single: Edelweiss 7%, diluted to 0.0002%, 0.0007%, 0.0021%
Single: Centella asiatica, 9%, diluted to 0.0005%, 0.0015%, 0.0045%
Combination: Gentian 15%+Edelweiss 7%, diluted to 0.0029%/0.0006% or 0.0084% %0.0018%
Combination: Gentian 15%+Edelweiss 7%+Centella 9%, diluted to 0.0009%/0.00021&/0.0001% or 0.0029%/0.0006%/0.0002% or 0.0084%/0.0018%/0.0005%

Gentian extract alone did not show a significant effect on MMP-1 inhibition in any of the concentrations tested.

Edelweiss and centella extracts, each individually, showed partially significant effects on MMP-1 inhibition in a concentration-dependent manner-however, both by increasing the inhibition, but also in the opposite sense, i.e. they increased the degradation of collagen 1.

The combinations of gentian and edelweiss extract showed significant synergistic effects on MMP-1 inhibition, while no such effects were detectable for both the combination of gentian and Centella and edelweiss and Centella.

The combinations of gentian, edelweiss and Centella extract showed significant or extremely significant effects on MMP-1 inhibition in all tested concentrations, which were even significantly higher than the effect of the positive standard (dexamethasone 1 μM) in the highest tested concentrations.

### CONCLUSION

These data support the conclusion that the extracts of gentian, edelweiss and centella are very suitable for delaying skin ageing. They have an influence on collagen 1 in the extracellular space, where an increase in collagen 1 expression was demonstrated. Furthermore, by influencing the enzyme MMP-1, which among other things catalyzes the cleavage of peptide bonds in proteins, they can delay the breakdown of collagen by inhibiting MMP-1.

In a suitable galenic formulation (W/O—O/W emulsion—cream, gel, lotion—oil, 2-phase or multi-phase product, micellar water, aqueous product with solubilizers) they are therefore suitable for supporting the collagen build-up processes, delaying the degradation processes of the same and consequently slowing down skin ageing and counteracting the formation of fine lines and wrinkles on the face. They are therefore particularly suitable for “anti-ageing”, “anti-wrinkle” and/or products that increase skin firmness.

1-12. (canceled)

13. A cosmetic composition comprising extracts of Gentiana, Leontopodium, and Centella, wherein the cosmetic composition comprises more extract in percentage by weight of Gentiana than Leontopodium or Centella.

14. An anti-ageing composition comprising the cosmetic composition according to claim 1.

15. An anti-wrinkle composition comprising the cosmetic composition according to claim 1.

16. An anti-pollution agent comprising the cosmetic composition according to claim 1.

17. A light stabilizer for non-therapeutic use, in particular a blue light and IR, UV stabilizer, comprising the cosmetic composition according to claim 1.

18. The cosmetic composition according to claim 1, wherein the extracts of Gentiana:Leontopodium:Centella are present in a ratio of 2.5-4:0.5-1:0.5-1 (w/w/w) in dry weight.

19. The cosmetic composition according to claim 1, wherein the extracts are obtained by an extracting agent from plant material or drug in the heat or cold, which contains water and/or at least one organic solvent, in particular C1-C4 alcohols or ethanol.

20. The cosmetic composition according to claim 1, wherein the extracts of Gentiana, Leontopodium, and Centella are each present in an amount of more than 0.5% by weight, in particular 1-2% by weight or 1-5% by weight in dry weight.

21. The cosmetic composition according to claim 1, further comprising an agent selected from the group consisting of thickener, humectant, solvent, stabilizer, emulsifier, skin-caring oil component, antioxidant, and fragrance.

22. The cosmetic composition according to claim 1, further comprising auxiliary substances and additives.

23. A method of using extracts of Gentiana, Leontopodium, and Centella in a cosmetic composition according to claim 18.

24. A method of non-therapeutically using extracts from Gentiana, Leontopodium, and Centella according to claim 1 as anti-ageing agents or anti-wrinkle agents or light protection agents or blue light and IR, UV protection agents.
